(19) United States
(12) Patent Application Publication
(10) Pub. No.: US 2021/0350874 A1
(43) Pub. Date: Nov. 11, 2021

(54) Methods for Analyzing Real Time Digital PCR Data

(71) Applicants: Yan Wang, San Diego, CA (US); Cory McCluskey, San Diego, CA (US)

(72) Inventors: Yan Wang, San Diego, CA (US); Cory McCluskey, San Diego, CA (US)

(21) Appl. No.: 17/316,666

(22) Filed: May 10, 2021

Related U.S. Application Data
(60) Provisional application No. 63/022,295, filed on May 8, 2020.

(51) Int. Cl. G16B 30/00

(52) U.S. Cl. CPC G16B 30/00

(57) ABSTRACT

Disclosed are methods for analyzing digital PCR data using real time measurements during the amplification cycles of the dPCR. An endpoint threshold is used to preliminarily separate positive amplifications from negative amplifications for a plurality of microreactions in the dPCR. The preliminary positive amplifications are further evaluated based on properties of the amplification curves of the microreactions so as to remove false positives.

### CROSS-REFERENCE TO RELATED APPLICATIONS

This application claims the benefit of provisional patent application No. 63/022,295, filed May 8, 2020, the content of which is incorporated by reference herein.

### FIELD OF THE INVENTION

This invention relates to analysis methods for detection of target nucleic acids, especially relates to an analysis method for using real time digital PCR for detecting target nucleic acids.

### BACKGROUND OF THE INVENTION

Polymerase chain reaction (PCR) is a method that uses a DNA polymerase and DNA polymerization reaction to generate thousands and millions of copies of a specific nucleic acid. It generally undergoes thermal cycles at different temperatures to repeatedly perform denaturing of double-stranded DNA, annealing of primers to target DNA sequences, and extending of primers to generate copies of the target sequence. PCR is an indispensible technique in molecular biology that is widely used to detect, identify, obtain and quantitate a DNA/RNA sequence of interest.

Quantitative PCR, also called real time qPCR, is a technique to quantitate the amount of a target sequence by monitoring the generation of the target sequence during the PCR amplification cycles. The production of the target sequence is monitored in real time either by a non-specific, fluorescent dyes that intercalate with any double stranded DNAs or by sequence-specific DNA probes that emit a detectable signal upon hybridization to a complimentary sequence. During a qPCR, the DNA-based fluorescence is measured at any time during the PCR cycles. The quantity of the target sequence is determined based on Cₜ, the threshold cycle number when the detected fluorescence level exceeds a threshold that is significantly above the background noise level. The relative quantification of gene expression can be determined by comparing the Cₜ of RNA/DNA from the target gene to the Cₜ of RNA/DNA from a house-keeping reference gene in the same sample. The absolute quantification is difficult and is usually based on creation of a standard curve with known DNA dilutions. Factors such as the variance of PCR amplification efficiency and non-exponential amplification can affect the accuracy of quantitative results and limit its ability to discriminate small fold-differences of gene quantities.

Digital PCR (dPCR) is a refinement of PCR technologies that allows absolute quantification of nucleic acid strands. The dPCR improves upon the conventional PCR by partitioning one PCR reaction into many small individual PCR microreactions such that each microreaction on average contains no more than one target nucleic acid molecule. Each microreaction approximately contains either 1 or 0 target nucleic acid molecule and gives a positive or negative binary readout at the end of PCR amplification. The fraction of positive readouts is determined and the absolute fraction of the target gene can be calculated based on Poisson statistical model. dPCR determines the absolute amount of the target nucleic acid by counting microreactions with the target molecules, which does not depend on the amplification cycle number and the comparison to a reference gene for quantification. By using massive amount of partitions, dPCR can be used to detect finer fold-differences than qPCR.

Since dPCR only concerns positive or negative readout from each microreaction, the dPCR is often performed by detecting the endpoint reaction products. However, the endpoint measurement lacks the real time kinetic information about the microreaction in each partition, which can provide valuable information for mechanistic investigation, assay optimization, and evaluation of false positives. Additionally, using the real time amplification information in PCR process can increase the dynamic range of PCR detection. The present invention provides a method for analyzing the real time dPCR amplification measurements of a large number of microreactions to provide more accurate evaluation of positive/false readouts of the dPCR.

### SUMMARY OF THE INVENTION

The present invention provides a method for analyzing digital PCR data using real time measurements during the amplification cycles of the dPCR. An endpoint threshold is used to preliminarily separate positive amplifications from negative amplifications for a plurality of microreactions in the dPCR. The preliminary positive amplifications are further evaluated based on properties of the amplification curves of the microreactions so as to remove false positives. Comparing to the endpoint dPCR analysis, the real time dPCR analysis allows for more sensitive, accurate and precise results, and provides greater linear range than that of the endpoint dPCR method.

In one embodiment, the present invention provides a method for analyzing digital polymerase chain reaction data, comprising the steps of: a) collecting readings from a plurality of microreactions during the dPCR amplification process; b) determining an amplification curve for each microreaction of the plurality of microreactions; c) determining a preliminary positive amplification or a preliminary negative dPCR amplification based on a threshold value; d) reevaluating the preliminary positive amplifications based on properties of amplification curves of the microreactions to obtain final determinations of positive amplifications; and e) counting the number of final positive and negative amplifications as the dPCR result.

In some embodiment, the readings collected from microreactions of the PCRs are fluorescent emission readings.

In some embodiment, the fluorescent emission readings are crosstalk calibrated when multiple fluorescent dyes are used.

In some embodiment, readings collected from the microreactions are normalized against a passive fluorescent dye.

In some embodiment, readings collected from the microreactions are normalized against the baseline readings of the same fluorescent dye.

In some embodiment, endpoint readings can be normalized readings, crosstalk calibrated readings, normalized and crosstalk calibrated readings, or raw readings.

In some embodiment, the amplification curve is generated from normalized readings, cross-talk calibrated readings, normalized and cross-talk calibrated readings, or raw readings.

In some embodiment, an amplification of a microreaction is determined to be a preliminary positive amplification when the endpoint reading of the microreaction is higher than a threshold value.

In some embodiment, the threshold value is an empirically determined value that separates the endpoint readings of positive amplifications from those of negative amplifications.

In some embodiment, the threshold value is set as following: plotting endpoint readings of the microreactions of the plurality of microreactions in a scatter plot; and selecting the threshold value that best separates the population of the endpoint normalized readings of positive amplifications from those of negative amplifications.

In some embodiment, the threshold value is set as following: generating a distribution curve of decreasing endpoint readings or ratios of endpoint readings at selected cycles; determining a threshold region within which the distribution curve has the steepest slope; and selecting the threshold value within the threshold region.

In some embodiment, the selection of an endpoint reading is based on properties of the amplification curves of the plurality of microreactions.

In some embodiment, the endpoint reading of a microreaction is selected from the last, the second last or the third last reading of the microreaction.

In some embodiment, the preliminary positive amplification is reevaluated based on inspection of the trend of the amplification curve.

In some embodiment, a preliminary positive amplification of a microreaction is determined to be false positive if the readings of the initial cycles of the microreaction are significantly higher than the baseline readings of the microreactions of the plurality of microreactions.

In some embodiment, a preliminary positive amplification of a microreaction is determined to be false positive if the cycle at which an amplification signal of the microreaction starts to exceed the baseline readings is significantly higher than the average cycle of the same for all the preliminary positive microreactions.

In some embodiment, wherein a preliminary positive amplification of a microreaction is determined to be false positive if the PCR cycle number at which an amplification signal of the microreaction starts to exceed the baseline readings is higher than 35.

In some embodiment, a preliminary positive amplification of a microreaction is determined to be false positive if the cycle at which an amplification signal of the microreaction starts to exceed the baseline readings is significantly lower than the average cycle of the same for all the preliminary positive microreactions.

In some embodiment, a preliminary positive amplification of a microreaction may be false positive if the last cycle reading is significantly lower than the maximum reading of the microreaction.

In another embodiment, the present invention provides a computer-implemented method of analyzing dPCR data, comprising: a) collecting readings of microreactions of a plurality of microreactions during the dPCR amplification process; b) determining and displaying amplification curves for microreactions of the plurality of microreactions; c) using a first parameter slider by a user to select a population of preliminary positive amplifications that satisfy the first parameter requirement; d) using a second parameter slider by a user to remove false positive amplifications that satisfy the second parameter requirement from the population of preliminary positive amplifications; and e) counting positive amplifications with the false positive amplifications removed as final positive amplifications.

In some embodiment of the computer-implemented method, the amplification curves are based on normalized readings, crosstalk calibrated readings, normalized and crosstalk calibrated readings, or raw readings.

In some embodiment of the computer-implemented method, the first parameter is an endpoint threshold, and wherein the first parameter requirement is that endpoint reading of a microreaction is higher than the endpoint threshold.

In some embodiment of the computer-implemented method, the second parameter is an initial value threshold, and wherein the second parameter requirement is that initial readings of a microreaction are higher than the initial value threshold.

In some embodiment of the computer-implemented method, the second parameter is a rising cycle number at which the amplification signal of a microreaction starts to exceed the baseline readings.

In some embodiment of the computer-implemented method, the second parameter requirement is that the rising cycle number is lower than a predetermined number.

In some embodiment of the computer-implemented method, the second parameter requirement is that the rising cycle number is higher than a predetermined number.

In some embodiment of the computer-implemented method, the second parameter is a threshold of ratio of endpoint readings of selected cycles, and the second parameter requirement is that the ratio of the endpoint readings of the selected cycles of a microreaction is lower than the threshold of the ratio of the endpoint readings of the selected cycles.

In some embodiment of the computer-implemented method, it further comprises removing an amplification curve of unexpected shape.

### BRIEF DESCRIPTION OF DRAWINGS

FIG. 1 is a flowchart illustrating an exemplary method of a real time dPCR analysis according to the invention.

FIG. 2 shows a scatter plot with randomly displayed normalized endpoint fluorescent emission readings for selection of a threshold value.

FIG. 3 shows an exemplary computer interface with a distribution curve of decreasing normalized endpoint readings, a control slider and an amplification plot.

FIG. 4 shows an exemplary computer interface with preliminary positive and negative amplification curves separation based on an endpoint threshold.

FIG. 5 shows an exemplary computer interface with preliminary positive and negative amplification curves separation based on an endpoint threshold and a ratio threshold.

FIGS. 6A-6D show positive amplification curves with false positives being sequentially removed. 6A, preliminary positive amplification curves with negative amplification curves removed; 6B, false positive amplification curves with high initial readings or abnormal shapes are removed; 6C, false positive amplification curves with early rising cycles are removed; and 6D, false positive amplification curves with late rising cycles are removed.

### DETAILED DESCRIPTION

Definitions: Unless otherwise defined, all technical and scientific terms used herein have the same meaning as commonly understood by one of the ordinary skills in the art to which this invention belongs.

The term “a” and “an” and “the” as used to describe the invention, should be construed to cover both the singular and the plural, unless explicitly indicated otherwise, or clearly contradicted by context. Similarly, plural terms as used to describe the invention, for example, nucleic acids, nucleotides and DNAs, should also be construed to cover both the plural and the singular, unless indicated otherwise, or clearly contradicted by context.

The term “digital PCR” or “dPCR”, as used herein, refers to a polymerase chain reaction technology that uses binary outputs of a large number of PCR amplifications to make absolute quantification of target nucleic acids. The target nuclear acids can be RNA or DNA sequences of interest. dPCR starts by partitioning a sample into many small individual compartments such that each compartment on average contains no more than one target sequence, and amplification reactions are then performed to determine the presence or absence of the target sequence in each compartment, where a positive and a negative amplification represents the presence and the absence of the target sequence, respectively. The fraction of compartments with the target sequence (p) is used to calculate the actual fraction of the target sequence in the sample (τ) based on Poisson statistic model where τ=−ln (1−p).

The term “real time digital PCR”, as used herein, refers to a digital PCR in which generation of the PCR products in microreactions of a plurality of microreactions is monitored during the PCR amplification cycles, in contrast to endpoint digital PCR, where only the PCR products at the end of PCR cycles are measured. In a real time digital PCR, the temporal data of the PCR amplification in microreactions of a plurality of microreactions can be used in evaluation of the binary output, thereby increasing the accuracy and fidelity of the dPCR results.

Digital PCR is performed by partitioning a sample into large quantities of small microreactions such that each microreaction on average contains no more than one target element, and PCR amplifications are then performed to determine the presence or absence of the target element in each microreaction. The readout in each microreaction is binary which only concerns whether it has or does not have the target element. The result of a dPCR is the number of positive amplifications and the number of negative amplifications (no amplifications) which can be converted to actual number of the target element in the sample. Most commercially available digital PCR machines only measure the endpoint PCR products after PCR amplifications are completed. However, the temporal data of PCR cycles that can provide valuable information for analysis of dPCR readouts are unavailable in these endpoint digital PCRs. The present invention provides a method for analyzing digital polymerase chain reaction data using real time measurements during the amplification cycles of the dPCR. An endpoint threshold is used to preliminarily separate positive amplifications from negative amplifications for a plurality of microreactions in the dPCR. The preliminary positive amplifications are further evaluated based on properties of the amplification curves of the microreactions so as to remove false positives. Comparing to the endpoint dPCR analysis, the real time dPCR analysis allows for more sensitive, accurate and precise results, and provides greater linear range than that of the endpoint dPCR method.

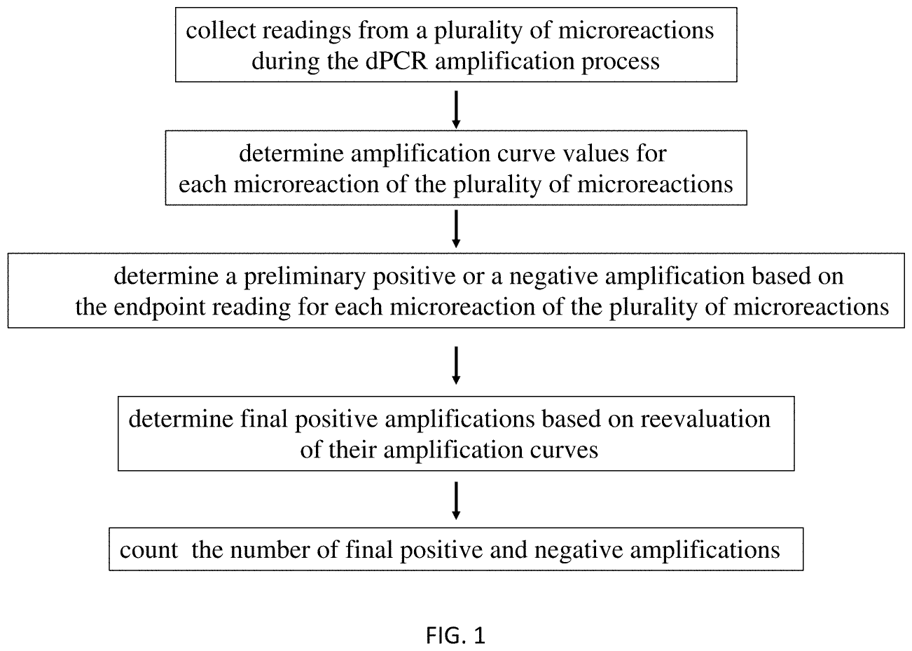

In one embodiment, the present invention provides a method for analyzing digital polymerase chain reaction data, comprising the steps of: a) collecting readings from a plurality of microreactions during the dPCR amplification process; b) determining amplification curve values for each microreaction of the plurality of microreactions; c) determining preliminary positive amplifications based on a threshold value; d) reevaluating the preliminary positive amplifications based on properties of amplification curves of the microreactions of the plurality of microreactions to obtain final determinations of positive amplifications; and e) counting the number of final positive and negative amplifications as dPCR result. FIG. 1 shows an exemplary flowchart of the real time dPCR analysis process.

To perform a real time digital PCR, a sample with PCR reagents is partitioned into large quantities (i.e. 20,000) of small compartments in a PCR chip for carrying out a large number of PCR microreactions in parallel. The dPCR chip is sent to a thermal cycler that is programed to run a number of thermal cycles specific for PCR requirements. For each PCR cycle, a copy of new target sequence is generated using existing target sequence as the template. The generation of the target sequence during the PCR cycles is monitored by a detection system that is coupled to the thermal cycler. A true amplification indicates the presence of a target sequence in a particular compartment. By counting the number of true amplifications, the number or the percentage of the target sequence in a sample can be calculated.

The generation of the target during the PCR cycles can be detected in many ways, including but not limited to, fluorescence detection, detection of positive or negative ions, pH detection, voltage detection, or current detection, alone or in combination. The most commonly used method of detection is fluorescence detection where generation of a target results in increase of fluorescent emission. Methods used for the detection of PCR products includes 1) using non-specific fluorescent dyes that intercalate with double-stranded DNA generated during PCR amplification and 2) using sequence-specific DNA probes labelled with a fluorescent reporter to hybridize to target sequences produced by PCR amplification. Fluorescent images of the plurality of microreaction sites can be captured and converted pixel by pixel to grey scale numbers which are used as fluorescent emission readings for microreactions. When using multiple fluorescent dyes in the same compartment, crosstalk (or bleedthrough) from one fluorescent emission to another can occur. In some embodiment, the crosstalk effect from one fluorescent dye to another may need to be calibrated. The crosstalk between two fluorophores can be corrected by a calibration constant. The crosstalk between fluorophore A and B is corrected as follows:

F_{A}′=F_{A}−K_{B->A}*F_{B}

F_{B}′=F_{B}−K_{A->B}*F_{A}

wherein F_{A} and F_{B} are raw fluorescence intensity of A and B, respectively; F_{A}′ and F_{B}′ are calibrated fluorescence intensity of A and B, respectively; K_{B->A} is the calibration constant for correcting bleedthrough from fluorescence channel B to A; and K_{A->B} is the calibration constant for correcting bleedthrough from fluorescence channel A to B.

A dPCR chip contains a large number of compartments for holding small volumes of samples for carrying out PCR microreactions. The compartments can be, for example, through-holes, wells, chambers, cavities, or indentations. A dPCR sample should be partitioned evenly into all the compartments in a chip. In practice, the volume partitioned among the chip compartments often varies from one unit to another, which contributes to variations in reading among different compartments. To compensate for the volume difference and other variations such as differences in shape, position and dye concentration, a raw reading collected from a signal detector can be further normalized. In a fluorescence based dPCR system, several reporter fluorescent dyes along with a passive fluorescent dye are employed to detect target generation. Emissions from reporter fluorescent dyes are directly correlated with PCR generation of nucleic acids while emission from the passive fluorescent dye is not related with generation of nucleic acids. In some embodiment, an emission reading of a reporter fluorescent dye can be divided against an emission reading of the passive fluorescent to obtain a normalized reporter reading. A single or an average reading of the passive fluorescent dye can be used in the normalization. In some embodiment, an emission reading of a reporter fluorescent dye can also be divided against the baseline readings of the same fluorescent dye to obtain a normalized reading. A baseline reading refers to an emission reading without detectable target amplification signal. A single or an average baseline reading of the reporter fluorescent dye can be used in the normalization.

A PCR amplification curve is plotted as fluorescence signals from each microreaction against the cycle number. The amplification curve shows the accumulation of product over the duration of the PCR process. A normal amplification curve has an S shape with a ground phase, an exponential phase, and a plateau phase. By inspecting the shape and values of an amplification curve, one can distinguish a true amplification curve from a false one. The fluorescence signal used in an amplification curve can be emission readings calibrated for crosstalk, normalized emission readings, crosstalk calibrated and normalized readings, or raw emission readings. It is preferred to use crosstalk calibrated and normalized readings to make the amplification curve, but amplification curves made of other types of readings can be acceptable under some circumstances. The fluorescence signal can be measured at an n-cycle interval (n=1, 2, 3, 4, 5, 6, 7, 8, 9, 10, 11, 12, 13, 14, 15, 16, 17, 18, 19, 20). For example, it can be measured at a 5-cycle interval which gives a total of 8 emission readings for a 40-cycle PCR experiment.

The present invention uses endpoint readings to select a population of preliminary positive amplifications in the plurality of microreactions. The endpoint reading should be an emission reading from late cycles when the PCR products are accumulated at a high level. It can be, for example, the last reading, the second last reading, and the third last reading. It can also be the maximum reading of a microreaction. The selection of an endpoint reading depends sometimes on the property of the amplification curve of a particular assay. For example, the reading of last PCR cycle may be lower than that of the second last cycle due to fluorescent dye quenching, and the reading of the second last cycle should be chosen as the endpoint reading. The endpoint reading can be emission readings calibrated for crosstalk, normalized emission readings, crosstalk calibrated and normalized readings, or raw emission readings. It is preferred to use crosstalk calibrated and normalized readings as the endpoint reading, but endpoint readings made of other types of readings can be acceptable under some circumstances.

If a target molecule is present in a compartment, a true PCR amplification will generate a large number of copies of the target molecule which increases the endpoint fluorescent signal to be higher than the baseline signals of the compartment. An endpoint threshold is used to select a population of preliminary positive amplifications from the negative amplifications. The microreactions having an endpoint reading higher than the endpoint threshold are selected as preliminary positive microreactions. The threshold can be empirically determined to be the one that best separates the population of positive amplifications from the population of negative amplifications. In some embodiment, the threshold is selected using a scatter plot in which the fluorescent emission readings of two reporter fluorescent dyes are plotted along two axes. A threshold can be chosen that best separates the positive cluster and the negative cluster. In some embodiment, a histogram of binned endpoint fluorescent emission readings can be used to select a threshold. A threshold can be chosen between a positive amplification peak and a negative amplification peak in the histogram. In some embodiment, all the endpoint fluorescent emission readings are randomly displayed on the same plot. The plot shows a population of potential positive amplifications dots separated from a population of negative amplification dots. A threshold can be selected in the region that best separates the two populations.

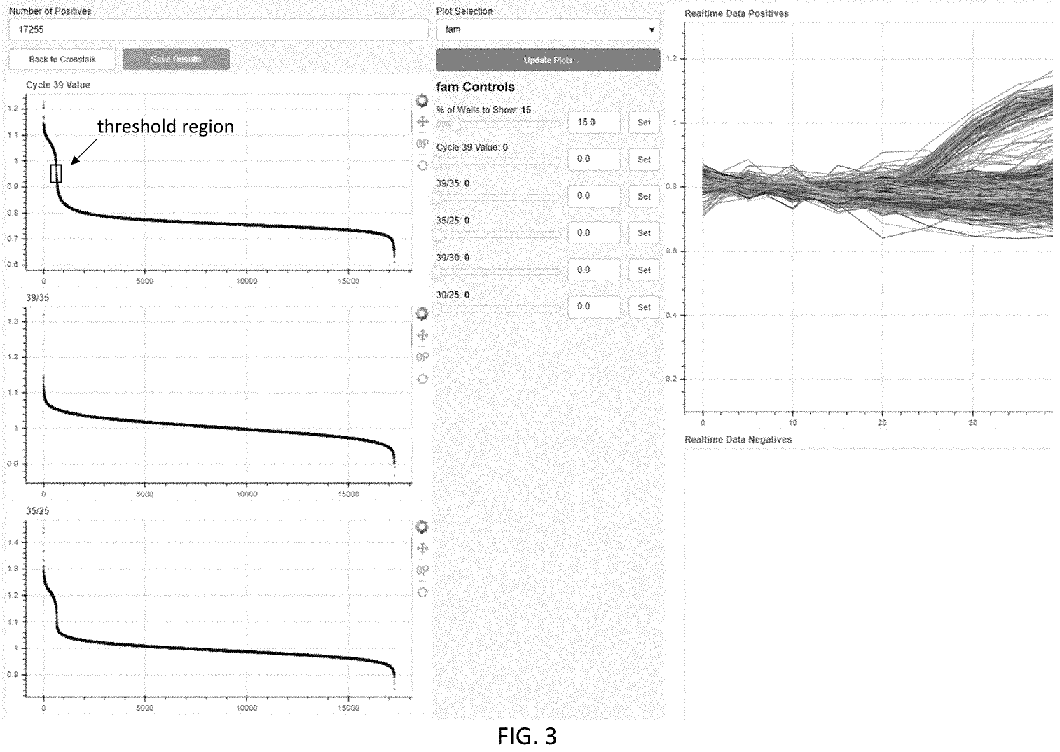

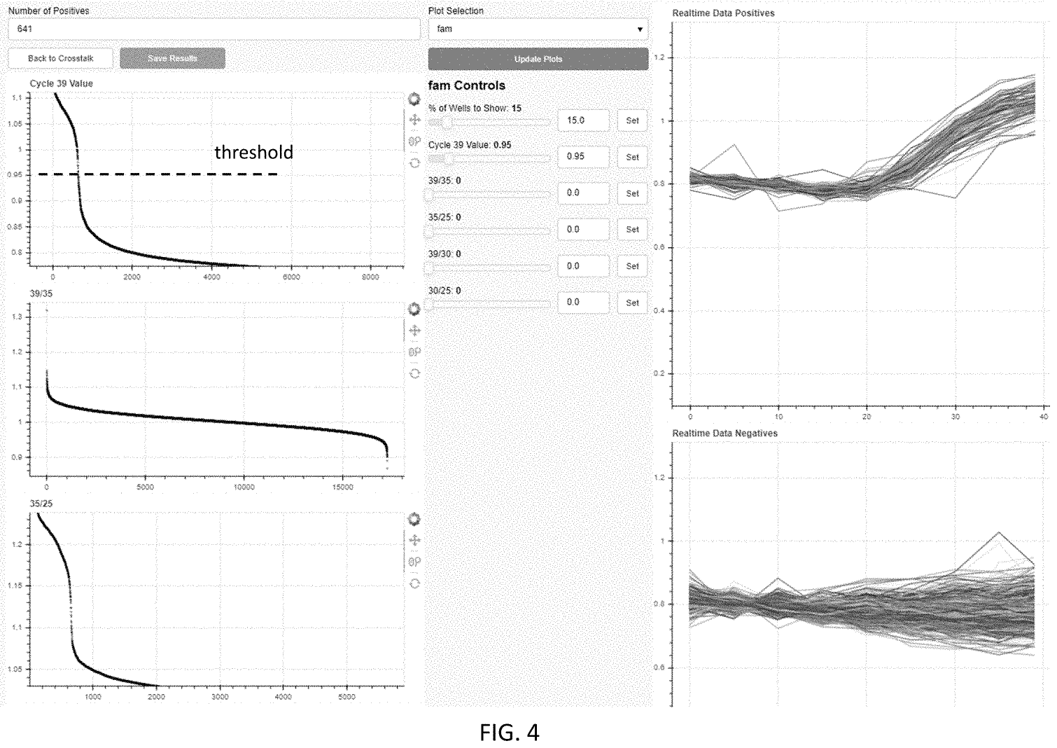

In some embodiment, a threshold value can be selected using a distribution curve of decreasing endpoint readings, where the normalized endpoint readings were plotted in a descending order in terms of the fluorescent emission intensity. The y-axis is the normalized endpoint fluorescent emission reading. The x-axis is the order number from 1 to n assigned to each endpoint reading based on the order of intensity of the endpoint emission reading. The endpoint reading with highest value of fluorescent emission intensity is assigned to the order number 1, the second highest endpoint reading is assigned to the order number 2, and it continues until all the endpoint readings are orderly assigned to respective order numbers. The distribution curve can be made for endpoint readings at a particular dPCR cycle (e.g. the last PCR cycle). A distribution curve of decreasing endpoint readings can be separated into a positive amplification region, a negative amplification region and a threshold region that is between the positive and the negative region. Compared to the positive and the negative region, the endpoint readings in the threshold region is less dense. The distances between adjacent endpoints in the threshold region are bigger than the distances between adjacent endpoints in a positive or negative region. The slope of the distribution curve in the threshold region is steeper than the slopes of the regions before and after the threshold region in the distribution curve. Excluding the endpoint readings at both ends of the distribution curve, the distribution curve in the threshold region has the steepest slope. In the exemplary computer interface, the threshold can be selected within the threshold region by manually setting a threshold value in the middle control panel and evaluating the separation of the positive and negative amplification groups in the right panel (FIG. 3). A threshold is selected within the threshold region, for example, the middle point of the threshold region, that best separates the positive from the negative amplification group. A lower threshold can be preferably selected because false positives can be removed with further inspection of the amplification curves. In FIG. 4, a threshold is selected at 0.95 and the preliminary positive and negative amplification curves are separately shown in the right panel.

In some embodiment, the distribution curve can be made of ratios of emission readings from two selected cycles in lieu of endpoint readings at a single cycle, and a threshold value can be selected as described above. For example, it is found that there is no detectable signal until PCR cycle 25 in a typical dPCR process, and the amplification signal at PCR cycle 35 is close to the maximum value. The ratio of emission readings from cycle 35 to cycle 25 can be used to make the distribution curve. The y-axis of the distribution curve is the ratio of emission readings of cycle 35 vs. cycle 25. The x-axis is the order number from 1 to n. The threshold of ratios of emission readings at cycle 35 and cycle 25 can be selected using the same method as described above. Using the threshold of the ratios of emission readings at cycle 35 and cycle 25 can help to remove false positives that have increased signals at cycle 25 or earlier. The ratio threshold can be used to independently select positive amplifications, or it can be used together with the endpoint threshold to determine positive amplifications.

Once a population of preliminary positive microreactions are selected by the threshold method, the amplification curves of the preliminary positive microreactions can be further evaluated to determine if they are true positive amplifications. In some embodiment, a preliminary positive amplification of a microreaction is determined to be false positive if the readings of the initial cycles of the microreaction are significantly higher than the baseline readings of the microreactions of the plurality of microreactions. Since a positive microreaction has only one copy or rarely two copies of target sequence, the initial PCR cycles (i.e. cycle 1-10) should have little or no detectable amplification signals. Elevated fluorescence signals in the initial cycles indicates a likely false positive, for example, a contaminated bright spot. The shape of an amplification curve can be used as another criterium to determine if a preliminary positive amplification is true or false one. A standard amplification curve has an S shape with a ground phase, an exponential phase and a plateau phase. An amplification curve deviated from the standard shape is likely to be a false positive. For example, an amplification curve with multiple peaks or linear type amplification curve is likely to be a false positive.

In some embodiment, a preliminary positive amplification of a microreaction is determined to be false positive if the cycle number at which an amplification signal of the microreaction starts to exceed the baseline readings, referred as a rising cycle, is significantly higher than the average cycle number of the same for all the preliminary positive microreactions. The average rising cycle for positive microreactions can be empirically determined. The average rising cycle for dPCR is generally between 20 to 28. For example, if a positive microreaction starts to have an amplification signal exceeding the baseline level at cycle 35, which is much higher than the expected value, the microreaction is likely to be a false positive. In some embodiment, a preliminary positive amplification of a microreaction is determined to be false positive if the rising cycle is significantly lower than the average cycle of the same for all the preliminary positive microreactions. For example, a positive microreaction with a rising cycle at 15 is likely to have a false positive. These methods help to remove false positive microreactions where the amplification signals occur too early or too late than expected cycle number. In some embodiment, preliminary positive amplification of a microreaction may be false positive if the last reading is significantly lower than the maximum reading of the microreaction. The last reading being slightly lower than the second last reading can sometimes happen due to, for example, fluorescent quenching. In this case, the amplification is not considered to be false positive. When the last reading is significantly lower than the maximum reading and the amplification curve is deviated from the standard shape of a normal amplification curve, it is reasonable to consider the amplification to be false positive.

In another embodiment, the present invention provides a computer-implemented method of analyzing dPCR data, comprising: a) collecting readings of microreactions of a plurality of microreactions during the dPCR amplification process; b) determining and displaying amplification curves for microreactions of the plurality of microreactions; c) using a first parameter slider by a user to select a population of preliminary positive amplifications that satisfy the first parameter requirement; d) using a second parameter slider by a user to remove false positive amplifications that satisfy the second parameter requirement from the population of preliminary positive amplifications; and e) counting positive amplifications with the false positive amplifications removed as true positive amplifications.

The present invention provides a visualization and analysis tool for user to manually change certain analysis parameters to select or remove positive amplification microreactions. It provides an interface displaying any selected amplification curves including those for all the microreactions of a dPCR. It also provides sliders for different parameters that a user can use to manually change the value of the parameter of interest, and visualize the results due to the change of the parameter. For example, it enables a user to choose an endpoint threshold and show all the amplification curves having endpoint readings higher than the endpoint threshold. A user can move the endpoint threshold slider to change the value of the threshold, and watch the change of positive amplification curves with the change of the threshold. This provides a visual tool for user to choose an appropriate threshold by human inspection.

In some embodiment of the computer-implemented method, the amplification curves are based on normalized readings, crosstalk calibrated readings, normalized and crosstalk calibrated readings, or raw readings.

In some embodiment, the analysis tool provides a slider of an endpoint threshold that can be changed by a user, and it displays the positive amplification curves of the microreactions having endpoint reading higher than the endpoint threshold.

In some embodiment, the analysis tool provides a slider of an initial value threshold that can be changed by a user, and it removes false positive microreactions with initial readings higher than the initial value threshold. By direct inspection of the removal of the false positive amplifications due to the change of the initial value threshold, a user can find an appropriate threshold value.

In some embodiment, the analysis tool provides a slider of a rising cycle number at which the amplification signal of a microreaction starts to exceed the baseline readings. User can set a minimum rising cycle number, and it removes false positive microreactions with a rising cycle number lower than the minimum rising cycle number.

In some embodiment, user can set a maximum rising cycle number, and it removes false positive microreactions with a rising cycle number higher than the maximum rising cycle number.

In some embodiment, a user can manually remove a positive amplification curve having an unexpected or abnormal shape. For example, a positive microreaction is considered to be a false positive if its last reading is significantly lower than the maximum reading. With this visual analysis tool, a user can manually change multiple parameters to select positive amplifications and remove false positives until a satisfactory result is achieved.

### EXAMPLES

### Example 1. Methods to Select a Threshold

This example illustrates two methods used to select a threshold. The raw fluorescence emission readings were normalized against a reference fluorescence emission reading. The last cycle readings were used as the endpoint readings.

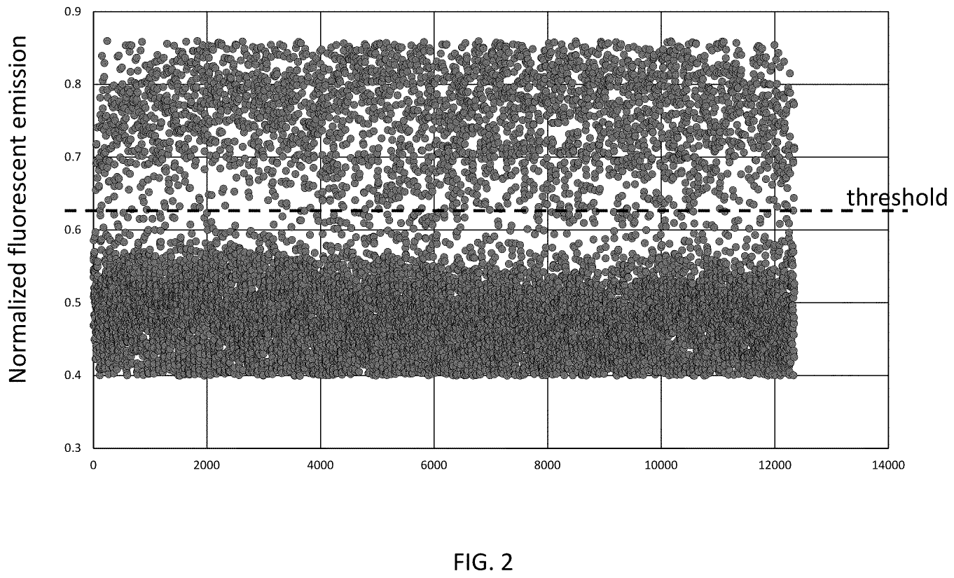

The first method used normalized endpoint readings that were randomly plotted on a scatter plot (FIG. 2). The y-axis is the normalized endpoint fluorescent emission reading. The x-axis is an order number ranging from 1 to n, randomly assigned to an endpoint emission reading, where n is the total number of all the valid emission readings in a dPCR experiment. The population of negative amplifications have lower endpoint readings and the population of positive amplifications have higher endpoint readings. A threshold of about 0.62 can be chosen to separate these two populations.

The second method for selection of a threshold value used a distribution curve of decreasing endpoint readings, where the normalized endpoint readings were plotted in a descending order in terms of the fluorescent emission intensity. The y-axis is the normalized endpoint fluorescent emission reading. The x-axis is the order number from 1 to n assigned to each endpoint reading based on the order of emission intensity of the endpoint emission reading. The distribution curve can be made for endpoint readings at a particular dPCR cycle (e.g. the last PCR cycle). In the computer interface of FIG. 3, a distribution curve is provided for cycle 39, the last PCR cycle of a real time dPCR assay. In the exemplary computer interface, the threshold can be selected within the threshold region by manually setting a threshold value in the middle control panel and evaluating the separation of the positive and negative amplification groups in the right panel (FIG. 3). In FIG. 4, a threshold is selected at 0.95 and the preliminary positive and negative amplifications are separated in the right panel.

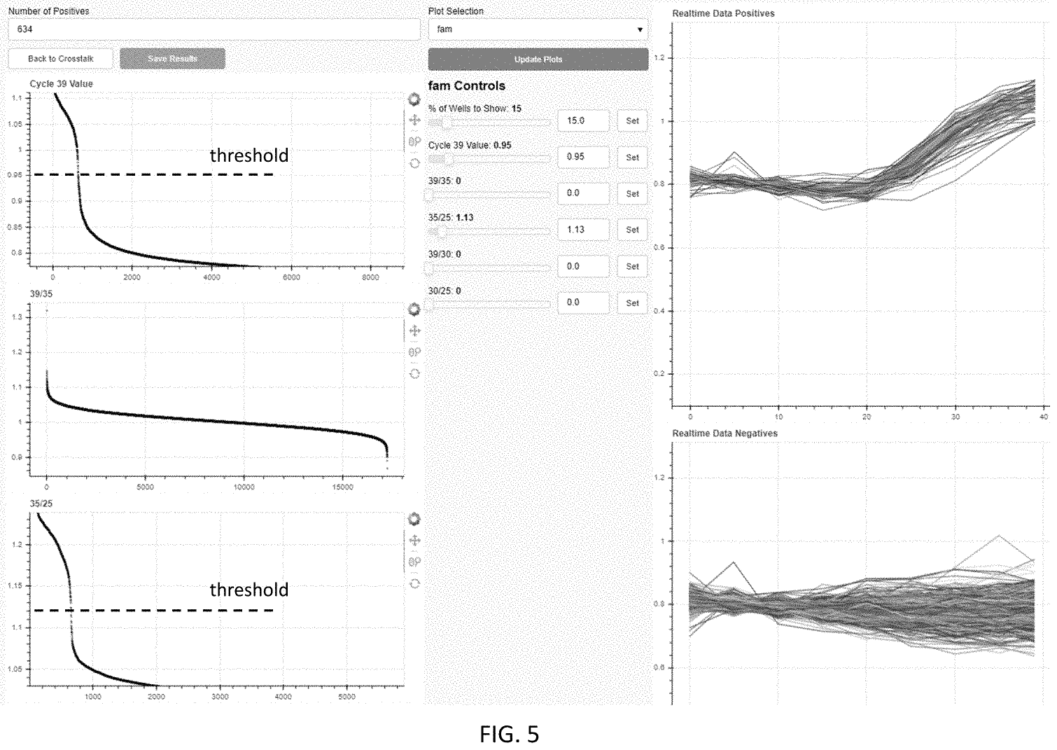

The distribution curve can also be made of ratios of emission readings from two selected cycles and a threshold can be selected as described above. For example, it is found that there is no detectable signal until PCR cycle 25 in a typical dPCR process, and the amplification signal at PCR cycle 35 is close to the maximum value. The distribution curve can be made of ratios of emission readings from cycle 35 to cycle 25. The threshold made from ratios of emission readings at cycle 35 and cycle 25 can be used to independently select positive amplifications, or it can be used together with the threshold of single endpoint readings to determine positive amplifications. Using the ratios of emission readings at cycle 35 and cycle 25 can help to remove false positives that have increased signals at cycle 25 or earlier. In FIG. 5, a threshold of endpoint readings at cycle 39 and a threshold at the ratios of emission readings at cycle 35 and cycle 25 are used together to determine positive amplifications. Using the threshold of the ratios of emission readings at cycle 35 and cycle 25 removed false positive amplifications from the preliminary positive amplifications that were selected by the endpoint threshold alone.

### Example 2. Methods to Remove False Positive Amplifications

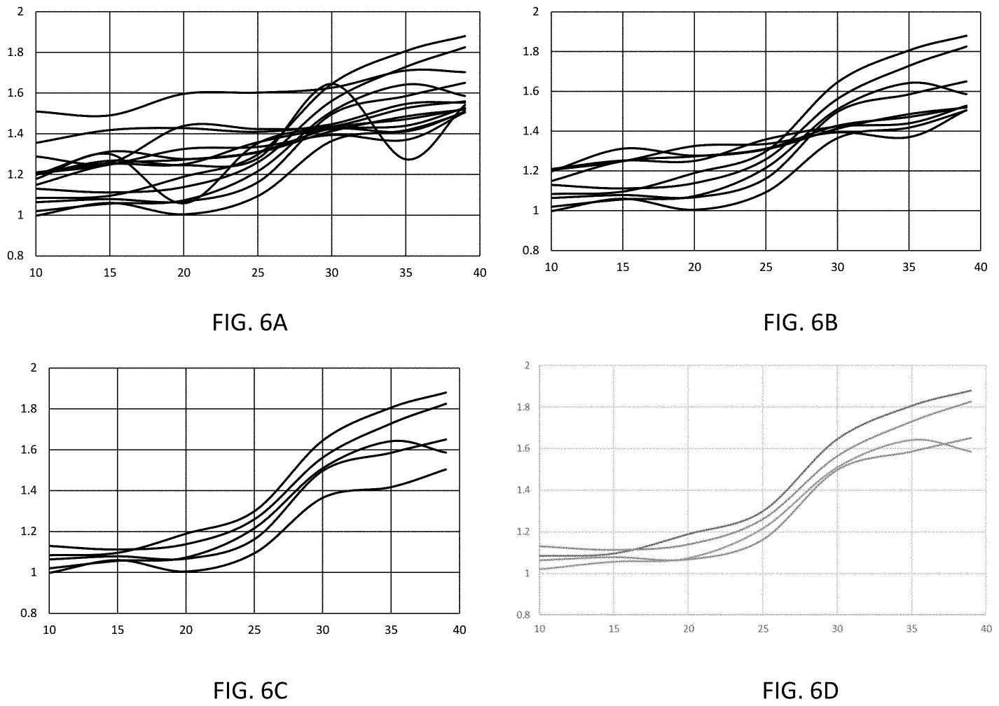

This example shows how to remove false positives from preliminarily selected positive amplification curves. The negative amplification curves were removed by an endpoint threshold selection method and preliminary positive amplification curves are shown in FIG. 6A, which contain true positive amplifications and false positive ones. In FIG. 6B, amplification curves with higher initial readings or abnormal shapes were removed. In FIG. 6C, amplification curves with too early rising cycle numbers were removed. It can be seen that the amplification curves with early rising cycle numbers do not have a standard shape of a normal amplification curve. In FIG. 6D, amplification curves with too late rising cycle numbers were removed. The remaining amplification curves are true positive amplification curves.

Using the methods to remove false positive amplifications, the real time dPCR can provide more accurate results than the endpoint dPCR. Table 1 shows a comparison of dPCR results using endpoint vs. real time dPCR to measure T790M mutation rate. It shows in the Table 1 that, compared to the endpoint dPCR, the real time dPCR results have smaller standard deviations (SD) and smaller coefficient variations (CV). The measured results of real time dPCR are also closer to the expected mutant allele frequency as compared to those of endpoint dPCR. The results indicate that the real time dPCR measurement is more accurate than that of the endpoint dPCR.

TABLE 1
Endpoint vs Real time Comparison for T790M Mutation Detection Assay

| Expected Mutant | | | | | | | | | |
|---|---|---|---|---|---|---|---|---|---|
| Allele Frequency | | 0.00% | | 0.03% | | 0.10% | | 1.00% | |
| Platform | | Endpoint | Real Time | Endpoint | Real Time | Endpoint | Real Time | Endpoint | Real Time |
| Mutant Allele | AVG | 0.04% | 0.02% | 0.10% | 0.05% | 0.20% | 0.13% | 1.29% | 1.09% |
| Frequency | STDEV | 0.03% | 0.02% | 0.06% | 0.02% | 0.08% | 0.04% | 0.20% | 0.16% |
| | CV | 73.34% | 106.88% | 57.95% | 37.06% | 39.66% | 33.88% | 15.39% | 14.83% |

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

What is claimed is:

1. A method for analyzing digital polymerase chain reaction (dPCR) data, comprising the steps of:
a) collecting readings from a plurality of microreactions during the dPCR amplification process;
b) determining amplification curve values for each microreaction of the plurality of microreactions;
c) determining preliminary positive amplifications of the plurality of microreactions based on a threshold value;
d) reevaluating the preliminary positive amplifications based on properties of amplification curves of the microreactions of the plurality of microreactions to obtain final determinations of positive amplifications; and
e) counting the number of final positive and negative amplifications as dPCR result.

2. The method of claim 1, wherein the readings are fluorescent emission readings.

3. The method of claim 2, wherein the fluorescent emission readings are crosstalk calibrated when multiple fluorescent dyes are used.

4. The method of claim 2, wherein readings collected from the microreactions are normalized against a passive fluorescent dye.

5. The method of claim 2, wherein readings collected from the microreactions are normalized against the baseline reading of the same fluorescent dye.

6. The method of claim 1, wherein endpoint readings can be normalized readings, crosstalk calibrated readings, normalized and crosstalk calibrated readings, or raw readings.

7. The method of claim 2, wherein the amplification curve is generated from normalized readings, cross-talk calibrated readings, normalized and cross-talk calibrated readings, or raw readings.

8. The method of claim 1, wherein the threshold is a threshold endpoint reading or a threshold ratio of endpoint readings at selected cycles.

9. The method of claim 8, wherein the threshold value is an empirically determined value that separates the endpoint readings of positive amplifications from those of negative amplifications.

10. The method of claim 8, wherein the threshold value is set as following:
a) plotting endpoint readings of the microreactions of the plurality of microreactions in a scatter plot; and
b) selecting the threshold value that best separates the population of the endpoint normalized readings of positive amplifications from those of negative amplifications.

11. The method of claim 8, wherein the threshold value is set as following:
a) generating a distribution curve of decreasing endpoint readings or ratios of endpoint readings at selected cycles;
b) determining a threshold region within which the distribution curve has the steepest slope; and
c) selecting the threshold value within the threshold region.

12. The method of claim 11, wherein the threshold of endpoint reading and the threshold of ratios of endpoint readings at selected cycles are used together to determine positive amplifications.

13. The method of claim 1, wherein the endpoint reading of a microreaction is selected from the last, the second last or the third last reading of the microreaction.

14. The method of claim 1, wherein the preliminary positive amplification is reevaluated based on inspection of the trend of the amplification curve.

15. The method of claim 1, wherein a preliminary positive amplification of a microreaction is determined to be false positive if the readings of the initial cycles of the microreaction are significantly higher than the baseline readings of the microreactions of the plurality of microreactions.

16. The method of claim 1, wherein a preliminary positive amplification of a microreaction is determined to be false positive if the cycle at which an amplification signal of the microreaction starts to exceed the baseline readings is higher than a predetermined number.

17. The method of claim 1, wherein a preliminary positive amplification of a microreaction is determined to be false positive if the cycle at which an amplification signal of the microreaction starts to exceed the baseline readings is higher than 35.

18. The method of claim 1, wherein a preliminary positive amplification of a microreaction is determined to be false positive if the cycle at which an amplification signal of the microreaction starts to exceed the baseline readings is lower than a predetermined number.

19. The method of claim 1, wherein a preliminary positive amplification of a microreaction may be false positive if the last cycle reading is significantly lower than the maximum reading of the microreaction.

20. A computer-implemented method of analyzing dPCR data, comprising:
a) collecting readings of microreactions of a plurality of microreactions during the dPCR amplification process;
b) determining and displaying amplification curves for microreactions of the plurality of microreactions;
c) using a first parameter slider by a user to select a population of preliminary positive amplifications that satisfy the first parameter requirement;
d) using a second parameter slider by a user to remove false positive amplifications that satisfy the second parameter requirement from the population of preliminary positive amplifications; and
e) counting positive amplifications with the false positive amplifications removed as final positive amplifications.

21. The method of claim 20, wherein the amplification curves are based on normalized readings, crosstalk calibrated readings, normalized and crosstalk calibrated readings, or raw readings.

22. The method of claim 20, wherein the first parameter is an endpoint threshold, and wherein the first parameter requirement is that endpoint reading of a microreaction is higher than the endpoint threshold.

23. The method of claim 20, wherein the second parameter is an initial value threshold, and wherein the second parameter requirement is that initial readings of a microreaction are higher than the initial value threshold.

24. The method of claim 20, wherein the second parameter is a rising cycle number at which the amplification signal of a microreaction starts to exceed the baseline readings.

25. The method of claim 24, wherein the second parameter requirement is the rising cycle number is lower than a predetermined number.

26. The method of claim 24, wherein the second parameter requirement is the rising cycle number is higher than a predetermined number.

27. The method of claim 20, wherein the second parameter is a threshold of ratio of endpoint readings of selected cycles, and wherein the second parameter requirement is that the ratio of the endpoint reading of the selected cycle of a microreaction is lower than the threshold of the ratio of the endpoint reading of the selected cycles.

28. The method of claim 20, further comprising removing an amplification curve of unexpected shape.
